(19) United States
(12) Patent Application Publication
(10) Pub. No.: US 2026/0294675 A1
(43) Pub. Date: Oct. 1, 2026

(54) THERMAL TRANSFER SYSTEMS AND METHODS OF USE THEREOF

(71) Applicant: Modular Thermal Technologies, LLC, Pompano Beach, FL (US)

(72) Inventor: Bradley Combs, Parkland, FL (US)

(21) Appl. No.: 19/574,535

(22) Filed: Mar. 23, 2026

Related U.S. Application Data
(60) Provisional application No. 63/777,279, filed on Mar. 25, 2025.

(51) Int. Cl. A61F 7/02; A01K 1/035

(52) U.S. Cl. CPC A61F 7/02; A01K 1/0353; A61F 2007/0244; A61F 2007/0268; A61F 2007/0279

(57) ABSTRACT

Thermal transfer systems and methods of use thereof are provided. The thermal transfer system comprises an outer housing, a first layer, a second layer, and a thermal transfer apparatus. The outer housing comprises a first side and a second side. The outer housing defines a cavity therein. The first layer is positioned in the cavity. The first layer has a first thickness of at least 0.5 inches. The second layer is positioned in the cavity. The thermal transfer apparatus is removably positioned in the cavity and intermediate the first layer and the second layer. The first layer is intermediate the thermal transfer apparatus and the first side.

### CROSS-REFERENCE TO RELATED APPLICATIONS

This application claims priority to U.S. Provisional Patent Application No. 63/777,279, filed on Mar. 25, 2025, the contents of which are hereby incorporated by reference into this specification.

### FIELD

The present disclosure relates to thermal transfer systems and methods of use thereof.

### BACKGROUND

Pet beds can be manufactured in various shapes and sizes to cater to the unique needs of different pets, from small cats to large dogs. A pet bed can provide support for joints, a cozy space for rest, and/or other therapeutic benefits. There are challenges configuring a pet bed for the therapeutic needs of a pet.

### SUMMARY

One aspect according to the present disclosure is directed to a thermal transfer system comprising an outer housing, a first layer, a second layer, and a thermal transfer apparatus. The outer housing comprises a first side and a second side. The outer housing defines a cavity therein. The first layer is positioned in the cavity. The first layer has a first thickness of at least 0.5 inches. The second layer is positioned in the cavity. The thermal transfer apparatus is removably positioned in the cavity and intermediate the first layer and the second layer. The first layer is intermediate the thermal transfer apparatus and the first side. The thermal transfer apparatus comprises a first container comprising a first wall, and a second wall connected to the first wall at ends of each wall, thereby defining a first enclosed cavity. The thermal transfer apparatus comprises at least four protrusions extending from the first wall into the first enclosed cavity towards the second wall. Each protrusion comprises a third wall defining a second enclosed cavity. The thermal transfer apparatus comprises a first thermal transfer media positioned within the first enclosed cavity and a second thermal transfer media positioned within each second enclosed cavity. The first thermal transfer media has a first freezing point lower than a second freezing point of the second thermal transfer media.

It will be understood that the inventions disclosed and described in this specification are not limited to the aspects summarized in this Summary. The reader will appreciate the foregoing details, as well as others, upon considering the following detailed description of various non-limiting and non-exhaustive aspects according to this specification.

### BRIEF DESCRIPTION OF THE DRAWINGS

The features and advantages of the examples presented herein, and the manner of attaining them, will become more apparent, and the examples will be better understood, by reference to the following description taken in conjunction with the accompanying drawings, wherein:

FIG. 1 is side cross-sectional view illustrating an example of a thermal transfer system according to the present disclosure;

FIG. 2 is a top view illustrating an example of a thermal transfer apparatus according to the present disclosure;

FIG. 3 is a cross-section view of the thermal transfer apparatus of FIG. 2 taken across lines 3-3;

FIG. 4 is top view illustrating an example of a thermal transfer apparatus comprising two modules according to the present disclosure;

FIG. 5 is top view illustrating an example of a thermal transfer apparatus comprising four modules according to the present disclosure;

FIG. 6 is a front perspective view illustrating an example of a thermal transfer system comprising a bolster according to the present disclosure;

FIG. 7 is an image illustrating an example of a layer comprising an egg-crate shape according to the present disclosure;

FIG. 8 is an image illustrating an example of a layer comprising a substantially planar shape according to the present disclosure; and

FIG. 9 is a front perspective view illustrating an example of a thermal transfer system according to the present disclosure.

Corresponding reference characters indicate corresponding parts throughout the several views. The exemplifications set out herein illustrate certain embodiments, in one form, and such exemplifications are not to be construed as limiting the scope of the appended claims in any manner.

### DESCRIPTION

Various examples are described and illustrated herein to provide an overall understanding of the structure, function, and use of the disclosed thermal transfer systems and methods of using the thermal transfer systems. The various examples described and illustrated herein are non-limiting and non-exhaustive. Thus, the invention is not limited by the description of the various non-limiting and non-exhaustive examples disclosed herein. Rather, the invention is defined solely by the claims. The features and characteristics illustrated and/or described in connection with various examples may be combined with the features and characteristics of other examples. Such modifications and variations are intended to be included within the scope of this specification. As such, the claims may be amended to recite any features or characteristics expressly or inherently described in, or otherwise expressly or inherently supported by, this specification. Further, Applicant reserves the right to amend the claims to affirmatively disclaim features or characteristics that may be present in the prior art. The various examples disclosed and described in this specification can comprise, consist of, or consist essentially of the features and characteristics as variously described herein.

Any references herein to “various examples”, “some examples”, “one example”, “an example”, “non-limiting examples”, or like phrases mean that a particular feature, structure, or characteristic described in connection with the example is included in at least one example. Thus, appearances of the phrases “in various examples”, “in some examples”, “in one example”, “in an example”, “in a non-limiting example”, or like phrases in the specification do not necessarily refer to the same example. Furthermore, the particular described features, structures, or characteristics may be combined in any suitable manner in an example or examples. Thus, the particular features, structures, or characteristics illustrated or described in connection with one example may be combined, in whole or in part, with the features, structures, or characteristics of another example or other examples without limitation. Such modifications and variations are intended to be included within the scope of the present examples.

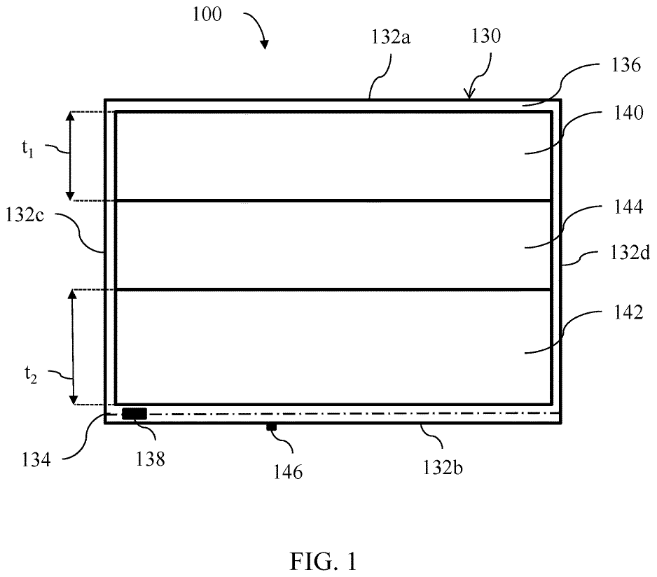

Referring to FIG. 1, a thermal transfer system 100 is provided. The thermal transfer system 100 comprises an outer housing 130, a first layer 140, a second layer 142, and a thermal transfer apparatus 144.

The housing 130 comprises a first side 132a and a second side 132b. The second side 132b may be positioned opposite the first side 132a. In various examples, the housing 130 can comprise a third side 132c and a fourth side 132d. It is understood that the housing 130 is illustrated as a rectangle for convenience but may take the form of other shapes, such as, for example, a circle, an oval, a parallelogram, a rhombus, and/or other shape. The sides 132a-132d may or may not be separated by distinct corners and may be portions of a singular surface in the case of an oval or other continuous surface.

The first side 132a can be configured to receive a user and provide thermal exchange with the user. For example, in the case where the thermal transfer apparatus 144 is cooled, the first side 132a can cool the user. In the case where the thermal transfer apparatus 144 is heated, the first side 132a can heat the user.

The user can be a pet, such as, for example, a dog or a cat.

The second side 132b can be configured to engage a surface, such as, for example, a floor. The second side 132b can comprise a slip resistant feature 146, such as, for example, a silicone pad.

The outer housing 130 defines a cavity 136 therein. The cavity 136 can be sized to receive the first layer 140, the second layer 142, the thermal transfer apparatus 144, and optionally an additional layer or layers. The housing 130 can support the orientation and/or positioning of the first layer 140, the second layer 142, the thermal transfer apparatus 144, and optionally an additional layer or layer relative to one another.

The outer housing 130 can be flexible and comfortable for a user. The outer housing 130 can protect the inner layers from dirt and/or spills. The outer housing 130 can comprise a fabric. In various examples, the fabric can be odor resistant and/or antimicrobial fabric.

The outer housing 130 and can be removable for changing colors, patterns, and/or washing. For example, the outer housing can comprise a resealable opening 134 that can be secured by a fastener 138. The fastener 138 can comprise a zipper, a hook and loop fastener, snaps, or a combination thereof.

The first layer 140 can be positioned in the cavity 136. The first layer 140 can provide comfort to a user of the thermal transfer system 100. In various examples, the first layer 140 abuts the first side 132a and the thermal transfer apparatus 144.

The first layer 140 can control thermal transfer between the thermal transfer apparatus 144 and the first side 132a. For example, the thermal transfer apparatus 144 can be cooled to a low temperature suitable for long durations of cooling. However, the low temperature may be too cold for a user and/or may cause the air surrounding the thermal transfer system 100 to cool too rapidly resulting in condensation on the thermal transfer system 100, which can be undesirable. The first layer 140 can provide suitable insulation such that the first side 132a does not become too cold or too hot, while enabling a desirable thermal transfer to provide therapy to a user in contact with the first side 132a.

The first layer 140 can comprise a desirable thickness, t₁, for comfort and/or controlling thermal transfer from the thermal transfer apparatus 144 to the first side 132a. For example, the thickness, t₁, can be 0.5 inch or greater, such as, for example, 0.75 inch or greater, 1 inch or greater, 1.25 inches or greater, or 1.5 inches or greater. The thickness, t₁, can be 3 inches or less, such as, for example, 2.5 inches or less, 2 inches or less, 1.75 inches or less, or 1.5 inches or less. In various examples, the thickness, t₁, can be in a range of 0.5 inch to 3 inches, such as, for example, 0.75 inches to 2.5 inches, or 1 inch to 2 inches.

The first layer 140 can exhibit a suitable thermal efficiency for thermal transfer between the thermal transfer apparatus 144 and the first side 132a. For example, the thermal efficiency of the first layer 140 can be 0.05 W/m*K or less, such as, for examples, 0.04 W/m*K or less, 0.03 W/m*K or less, or 0.025 W/m*K or less. The thermal efficiency of the first layer 140 can be 0.01 W/m*K or greater, such as, for example, 0.015 W/m*K or greater, or 0.2 W/m*K or greater. In various examples, the thermal efficiency of the first layer 140 can be in a range of 0.01 W/m*K to 0.05 W/m*K, such as, for example, 0.015 W/m*K to 0.04 W/m*K, or 0.02 W/m*K to 0.03 W/m*K. Thermal efficiency can be measured according to ASTM C518-21.

The R-value in metric (SI) units (RSI) of the first layer 140 can be in a range of 0.5 m²*K/W to 10 m²*K/W, such as, for example, 0.5 m²*K/W to 5 m²*K/W, 0.5 m²*K/W to 3 m²*K/W, or 1 m²*K/W to 3 m²*K/W.

The first layer 140 can comprise polyurethane, polyester, cotton, or a combination thereof. The first layer 140 can comprise foam, batting, or a combination thereof. In various examples, the first layer 140 can comprise a polyurethane foam. In certain examples, the first layer 140 can comprise batting comprising polyester, cotton, or a combination thereof.

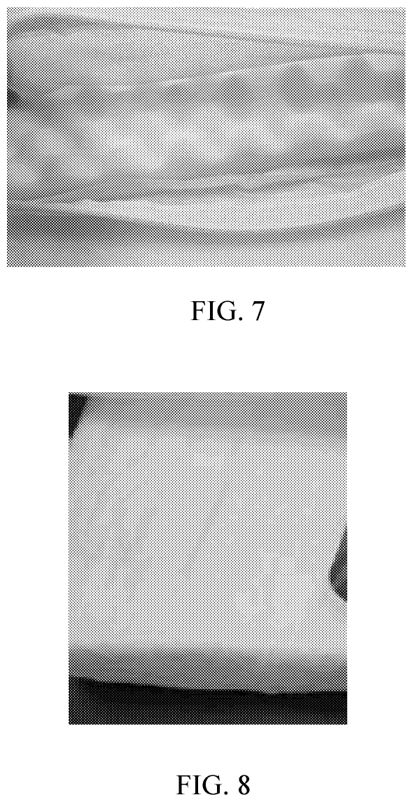

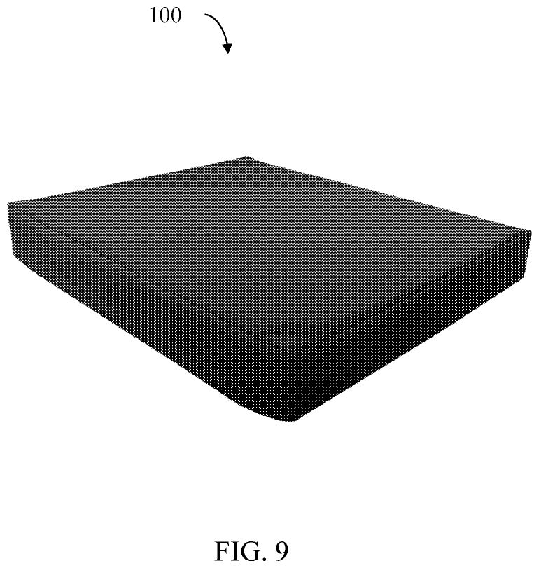

The first layer 140 can comprise an egg-crate shape (e.g., FIG. 7), a substantially planar shape (e.g., FIG. 8), or other shape as desired.

Referring again to FIG. 1, the second layer 142 can be positioned in the cavity 136. The second layer 142 can provide comfort to a user of the thermal transfer system 100. In various examples, the second layer 142 abuts the second side 132b and the thermal transfer apparatus 144.

The second layer 142 can insulate the thermal transfer apparatus 144 from the second side 132b. For example, the second layer 142 can enable the thermal transfer apparatus 144 to maintain a desirable temperature for an enhanced duration of time.

The second layer 142 can comprise a desirable thickness, t₂, for comfort and/or insulation. The thickness, t₂, can be greater than the thickness t₁, such as, for example, 0.5 inch or greater, 1 inch or greater, or 1.5 inches or greater. For example, the thickness, t₂, can be 2 inches or greater, such as, for example, 2.5 inches or greater, 3 inches or greater, or 3.5 inches or greater. The thickness, t₂, can be 4 inches or less, such as, for example, 3.5 inches or less, or 3 inches or less. In various examples, the thickness, t₂, can be in a range of 2 inches to 4 inches, such as, for example, 2 inches to 3 inches, or 3 inches to 4 inches.

The second layer 142 can exhibit a suitable thermal efficiency to insulate the thermal transfer apparatus 144 from the second side 132b. For example, the thermal efficiency of the second layer 142 can be 0.05 W/m*K or less, such as, for examples, 0.04 W/m*K or less, 0.03 W/m*K or less, or 0.025 W/m*K or less. The thermal efficiency of the second layer 142 can be 0.01 W/m*K or greater, such as, for example, 0.015 W/m*K or greater, or 0.2 W/m*K or greater. In various examples, the thermal efficiency of the second layer 142 can be in a range of 0.01 W/m*K to 0.05 W/m*K, such as, for example, 0.015 W/m*K to 0.04 W/m*K, or 0.02 W/m*K to 0.03 W/m*K.

The RSI value of the second layer 142 can be in a range of 1 m²*K/W to 20 m²*K/W, such as, for example, 1.25 m²*K/W to 15 m²*K/W, 1.5 m²*K/W to 10 m²*K/W, or 2 m²*K/W to 10 m²*K/W.

The second layer 142 can comprise polyurethane, polyester, cotton, or a combination thereof. The second layer 142 can comprise foam, batting, or a combination thereof. In various examples, the second layer 142 can comprise a polyurethane foam. In certain examples, the second layer 142 can comprise batting comprising polyester, cotton, or a combination thereof. The second layer 142 can comprise the same composition as the first layer 140 or a different composition.

The second layer 142 can comprise an egg-crate shape (e.g., FIG. 7), a substantially planar shape (e.g., FIG. 8), or other shape as desired. The second layer 142 can comprise the same shape as the first layer 140 or a different shape.

Referring yet again to FIG. 1, the thermal transfer apparatus 144 can be removably positioned in the cavity 136 and intermediate the first layer 140 and the second layer 142. The first layer 140 can be intermediate the thermal transfer apparatus 144 and the first side 132a and the second layer 142 can be intermediate the thermal transfer apparatus 144 and the second side 132b. In various examples, at least two thermal transfer apparatus, or at least three thermal transfer apparatus may be removably positioned in the cavity 136 intermediate the first layer 140 and the second layer 142.

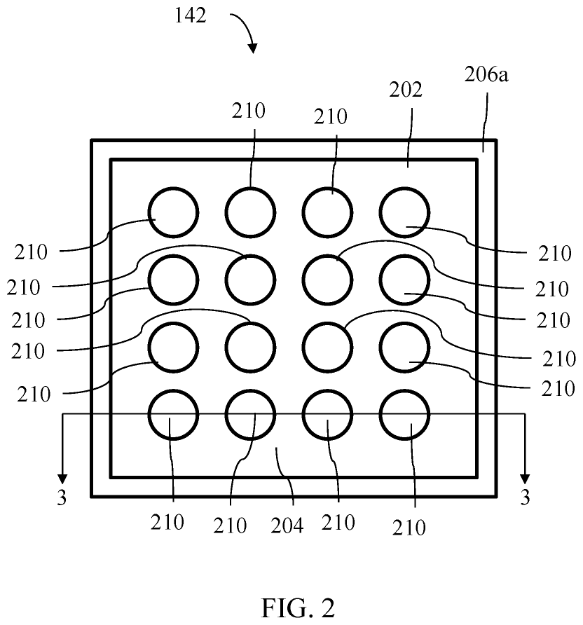

The thermal transfer apparatus 144 can be various types and/or shapes of thermal transfer apparatus. The thermal transfer apparatus 144 is described with references to the example shown in FIG. 2 for clarity and should not be limited thereto.

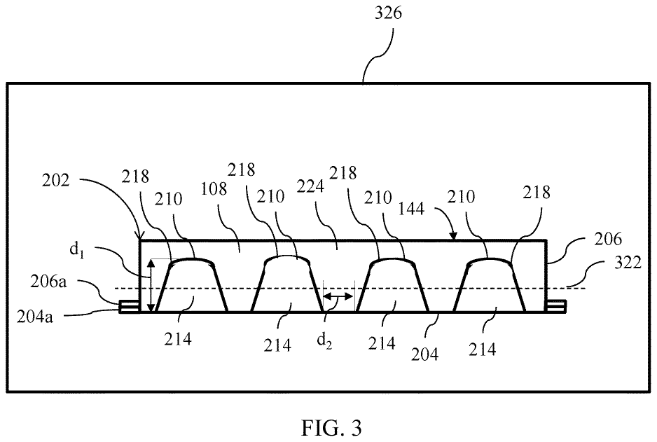

Referring to FIG. 2, the thermal transfer apparatus 144 can comprise a first container 202 and at least four protrusions 210, such as, for example, 16 protrusions as shown in FIG. 2 or other amount of protrusions (not shown). The first container 202 comprises a first wall 204 and a second wall 206. The second wall 206 is connected to the first wall 204, thereby defining a first enclosed cavity 224. For example, referring to FIG. 3, the second wall 206 can comprise edges 206a and the first wall 204 can comprise edges 204a. The edges 206a and 204a can be fused together by, for example, heat sealing or other adhesive technique such that the first wall 204 is fused to the second wall 206.

The protrusions 210 can extend from the first wall 204 into the first enclosed cavity 224 towards the second wall 206 a distance, d₁. The distance, d₁, can be in a range of 0.75 inches to 1.5 inches, such as, for example, 0.8 inch to 1.25 inches, 0.9 inch to 1.2 inches, or 0.95 inches to 1.2 inches. In various examples, the protrusions 210 can be integral with the first wall 204.

As illustrated in FIG. 3, the protrusions 210 can comprise a rounded cross-section in a plane (e.g., plane 322 in FIG. 3, which is extending out of the page) parallel to the first wall 204. In various examples, the protrusions 210 can comprise a different shape. Referring to FIG. 3, each protrusion 210 can taper away from the first wall 204. For example, a diameter of the rounded cross-section of each protrusion 210 can decrease as one moves away from the first wall 204 towards the second wall 206. The protrusions 210 can comprise the same shape or different shapes.

In certain examples, the protrusions 210 can be arranged in a grid, such as, for example, four rows of four columns as illustrated in FIG. 2, five rows of five columns, or eight rows of eight columns, or other configuration. Each protrusion 210 can be spaced away from an adjacent protrusion a substantially uniform distance. For example, referring to FIG. 3, a distance, d₂, between each protrusion 210 can be in a range of 0.1 inches to 1.25 inches, such as, for example, 0.3 inches to 1.25 inches, 0.5 inches to 1.1 inches, or 0.7 inches to 1.1 inches.

Each protrusion 210 comprises a third wall 218 defining a second enclosed cavities 214. In various examples, the third walls 218 are fused to the first wall 204 by, for example, heat sealing. The first wall 204, the second wall 206, and the third walls 218, each can comprise a thickness in a range of 0.5 mm to 5 mm such that the thermal transfer apparatus can remain pliable. In various examples, the first wall 204, the second wall 206, and the third walls 218 each can comprise plastic and may be substantially transparent.

A first thermal transfer media is positioned within the first enclosed cavity 224. A second thermal transfer media is positioned within each second enclosed cavity 214. The first thermal transfer media has a first freezing point lower than a second freezing point of the second thermal transfer media. For example, the first freezing point is lower than the second freezing point by at least 3 degrees Celsius, such as, for example, at least 5 degrees Celsius, or at least 8 degrees Celsius. The difference in freezing points enables the first thermal transfer media to be in a liquid state such that the thermal transfer apparatus is pliable while the second thermal transfer media can be in a solid state. Additionally, maintaining the first thermal transfer media in a liquid state can more efficiently maintain a temperature of the thermal transfer device while applied to a portion of a body of a patient.

The second freezing point can be no lower than temperatures found in conventionally available freezers so that the second thermal transfer media can be frozen in the conventionally available freezers. For example, the second freezing point can be in a range of −5 degrees Celsius to 5 degrees Celsius. The first freezing point can be lower than temperatures found in conventionally available freezer so that the first thermal transfer media may not freeze or the first freezing point may be configured to thaw efficiently at ambient temperature (e.g., 20 degrees Celsius +/−2 degrees Celsius). For example, the first freezing point can be in a range of −15 degrees Celsius to 0 degrees Celsius.

The first thermal transfer media and the second thermal transfer media, individually, can comprise water, glycol (e.g., propylene glycol), gelatin, a hydrogel, an additive, or a combination thereof. For example, the first thermal transfer media can comprise water and glycol and the second thermal transfer media can comprise water. In certain examples, the first thermal transfer media can comprise water, glycol, and gelatin and the second thermal transfer media can comprise water and gelatin.

The first thermal transfer media and/or second thermal transfer media can further comprise a dye. For example, the first thermal transfer media can be pink and the second thermal transfer media can be blue.

In various examples, referring to FIG. 3, the thermal transfer apparatus 144 can be positioned within a bag 326. The bag 326 can be substantially water impermeable to inhibit condensation from leaking into the cavity 136.

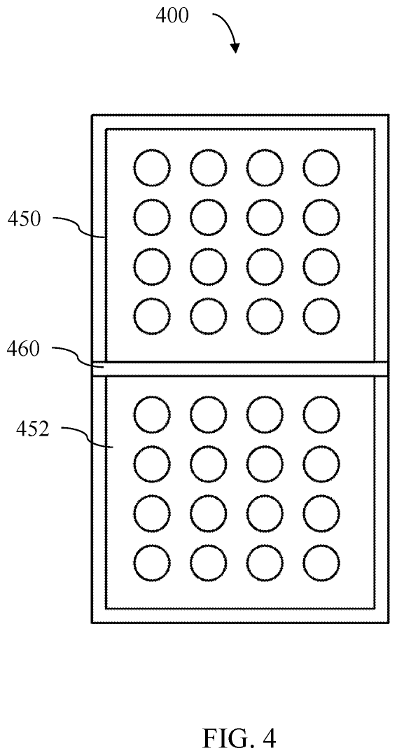

The thermal transfer apparatus 144 can comprise at least two modules. For example, referring to FIG. 4, a thermal transfer apparatus 400 is provided with two modules 450 and 452. Each module 450 and 452 can be the same or different. For example, the modules 450 and 452 can be configured the same as the thermal transfer apparatus 144. The modules 450 and 452 can be connected together at their edges 460. In various examples, the first walls of modules 450 and 452 can be part of a single continuous piece through all modules 450 and 452 and the second walls of modules 450 and 452 can be part of a single continuous piece through all modules 450 and 452. The edges 460 can be portions of the thermal transfer apparatus 400 that are fused together by, for example, a heat sealing or other adhesive technique such that the first wall is fused to the second wall.

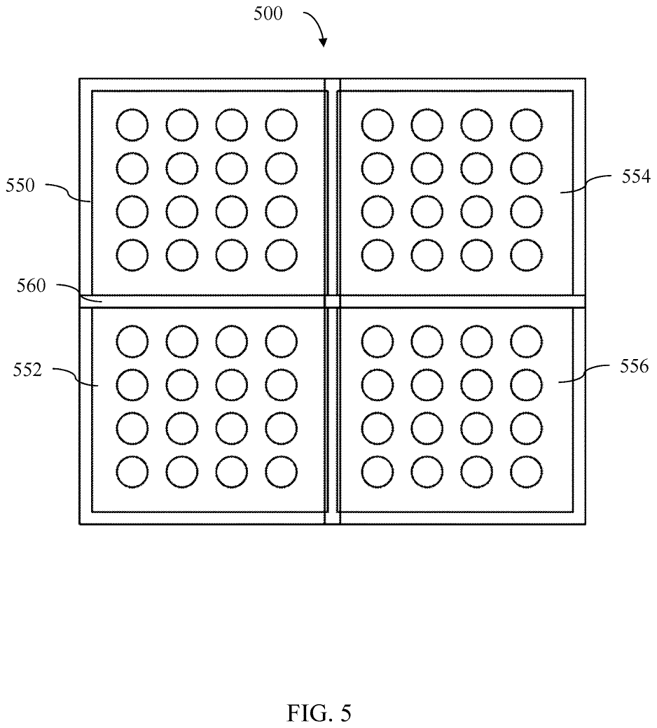

Referring to FIG. 5, a thermal transfer apparatus 500 is provided with four modules 550, 552, 554, and 556 in a first configuration. Each module 550, 552, 554, and 556 can be the same or different. For example, the modules 550, 552, 554, and 556 can be configured the same as the thermal transfer apparatus 144. The modules 550, 552, 554, and 556 are connected together at edges 560. In various examples, the first walls of modules 550, 552, 554, and 556 can be part of a single continuous piece through all modules 550, 552, 554, and 556 and the second walls of modules 550, 552, 554, and 556 can be part of a single continuous piece through all modules 550, 552, 554, and 556. The edges 560 can be portions of the thermal transfer apparatus 500 that are fused together by, for example, a heat sealer such that the first wall is fused to the second wall.

In various examples, the thermal transfer system 100 can comprise a thermal transfer apparatus as described in U.S. patent application Ser. No. 18/647,928 filed Apr. 26, 2024, U.S. patent application Ser. No. 18/647,928 filed Apr. 26, 2024, U.S. patent application Ser. No. 18/488,477 filed Oct. 17, 2023, U.S. patent application Ser. No. 29/878,766 filed Jun. 27, 2023, and U.S. Provisional Patent No. 63/379,969 filed Oct. 18, 2022, each of which is hereby incorporated by reference.

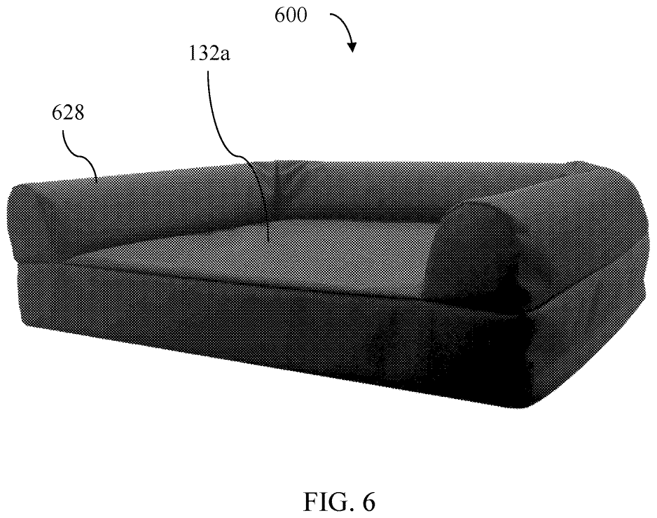

In certain examples, referring to FIG. 6, a thermal transfer system 600 is provided. The thermal transfer system 600 can be configured the same as the thermal transfer system 100. The thermal transfer system 600 can comprise a bolster 628 disposed on the first side 132a. The bolster 628 can support a user of the thermal transfer system 600.

Referring back to FIG. 1, the thermal transfer system 100 can be configured to maintain to maintain a temperature on the first side of no greater than 75 degrees Fahrenheit for at least 4 hours when an ambient temperature is 85 degrees Fahrenheit and the thermal transfer apparatus was stored in a freezer with a temperature in a range of 5 degrees Fahrenheit to 25 degrees Fahrenheit for at least 12 hours.

In various examples, the thermal transfer system 100 can be configured to maintain to maintain a temperature on the first side 132a of no greater than 70 degrees Fahrenheit for at least 8 hours when ambient temperature is 90 degrees Fahrenheit and the thermal transfer apparatus was stored in a freezer with a temperature in a range of 5 degrees Fahrenheit to 20 degrees Fahrenheit for at least 12 hours.

In various examples, the thermal transfer system 100 can be configured to maintain to maintain a temperature on the first side 132a of no greater than 75, 70, or 65 degrees Fahrenheit for at least 4, 6, 8, 10, or 12 hours when ambient temperature is 85, 90, or 95 degrees Fahrenheit and the thermal transfer apparatus was stored in a freezer with a temperature in a range of 5 to 20 or 5 to 25 degrees Fahrenheit for at least 12 hours.

In various examples, the thermal transfer system 100 can comprise additional layers in the cavity 136 as desired for the application. In certain examples, the first layer 140 and/or the second layer 142 can be in a bag, which may be moisture resistant.

The present disclosure provides a method for using the thermal transfer system 100. The method comprises disposing the thermal transfer apparatus 144 of the thermal transfer system 100 in a freezer to cool the thermal transfer apparatus 144 to a temperature of less than or equal to the second freezing point, thereby forming a cooled thermal transfer apparatus. The second thermal transfer media can freeze and the first thermal transfer media may not freeze depending on the first freezing point and the temperature of the freezer. The thermal transfer apparatus 144 can be removed from the freezer and disposed in the cavity 136 of the outer housing 130 so that the thermal transfer apparatus 144 can participate in a thermal transfer with the first side 132a. For example, heat can be absorbed through the first side 132a and the first layer 140 by the transfer of kinetic energy from particle to particle. When heat is absorbed by the first thermal transfer media, the first thermal transfer media may have a non-uniform temperature (e.g., first thermal transfer media closer to the first wall 204 and/or second wall 206 may be warmer than first thermal transfer media further away from the first wall 204 and/or the second wall 206). If the first thermal transfer media has a varying temperature, the density also varies causing circulation and movement of liquid portions of the first thermal transfer media. The warmed first thermal transfer media can pass by and contact the third wall 218 and heat can be absorbed there through into the second thermal transfer media.

The method can comprise cooling an object placed on the first side 132a or heating an object placed on the first side 132a.

The following numbered clauses are directed to various non-limiting embodiments and aspects according to the present disclosure.

Clause 1. A thermal transfer system comprising: an outer housing comprising a first side and a second side, the outer housing defining a cavity therein; a first layer positioned in the cavity, wherein the first layer has a first thickness of at least 0.5 inches and exhibits a thermal efficiency of no greater than 0.05 w/m*K; a second layer positioned in the cavity; and a thermal transfer apparatus removably positioned in the cavity and intermediate the first layer and the second layer, wherein the first layer is intermediate the thermal transfer apparatus and the first side, the thermal transfer apparatus comprising: a first container comprising a first wall, and a second wall connected to the first wall at ends of each wall, thereby defining a first enclosed cavity, at least four protrusions extending from the first wall into the first enclosed cavity towards the second wall and each protrusion comprising a third wall defining a second enclosed cavity, a first thermal transfer media positioned within the first enclosed cavity, and a second thermal transfer media positioned within each second enclosed cavity, wherein the first thermal transfer media has a first freezing point lower than a second freezing point of the second thermal transfer media.

Clause 2. The system of clause 1, wherein the first layer exhibits a thermal efficiency of no greater than 0.04 w/m*K.

Clause 3. The system of clause 1, wherein the first layer exhibits a thermal efficiency of no greater than 0.03 w/m*K.

Clause 4. The system of clause 1, wherein the first layer exhibits a thermal efficiency of no greater than 0.025 w/m*K.

Clause 5. The system of any of clauses 1-4, wherein the second layer exhibits a thermal efficiency of no greater than 0.05 w/m*K.

Clause 6. The system of any of clauses 1-5, wherein the second layer comprises a second thickness and the second thickness is greater than the first thickness.

Clause 7. The system of any of clauses 1-6, wherein the second thickness is in a range of 2 inches to 4 inches.

Clause 8. The system of any of clauses 1-7, wherein the first thickness is in a range of 1 inch to 3 inches.

Clause 9. The system of any of clauses 1-8, wherein the first layer comprises an egg-crate shape or a substantially planar shape.

Clause 10. The system of any of clauses 1-9, wherein the second layer comprises an egg-crate shape or a substantially planar shape.

Clause 11. The system of any of clauses 1-10, wherein the first layer and the second layer, individually, comprise polyurethane, polyester, cotton, or a combination thereof.

Clause 12. The system of any of clauses 1-11, wherein the first layer and the second layer, individually, comprise foam, batting, or a combination thereof.

Clause 13. The system of any of clauses 1-12, wherein the second side comprises at least one slip resistant feature.

Clause 14. The system of any of clauses 1-13, wherein the outer housing comprises fabric.

Clause 15. The system of any of clauses 1-14, wherein the outer housing comprises a zipper, a hook and loop fastener, snaps, or a combination thereof.

Clause 16. The system of any of clauses 1-15, wherein the thermal transfer apparatus is positioned within a bag.

Clause 17. The system of any of clauses 1-16, wherein the at least four protrusions are arranged in a grid.

Clause 18. The system of any of clauses 1-17, further comprising sixteen protrusions including the at least four protrusions, wherein the sixteen protrusions are arranged in four rows and four columns to form a first module.

Clause 19. The system of clause 18, further comprising at least two modules, including the first module and a second module.

Clause 20. The system of any of clauses 1-19, wherein the first freezing point is lower than the second freezing point by at least 3 degrees Celsius.

Clause 21. The system of any of clauses 1-19, wherein the first freezing point is lower than the second freezing point by at least 5 degrees Celsius.

Clause 22. The system of any of clauses 1-21, wherein the second freezing point is in a range of −5 degrees Celsius to 5 degrees Celsius.

Clause 23. The system of any of clauses 1-22, wherein the first thermal transfer media and the second thermal transfer media, individually, comprises water, glycol, gelatin, a hydrogel, an additive, or a combination thereof.

Clause 24. The system of any of clauses 1-23, wherein the first wall, the second wall, and the third wall, each comprise plastic.

Clause 25. The system of any of clauses 1-24, wherein the first wall, the second wall, and the third wall, each comprise a thickness in a range of 0.5 mm to 5 mm.

Clause 26. The system of any of clauses 1-25, wherein the system is configured to maintain a temperature on the first side of no greater than 75 degrees Fahrenheit for at least 4 hours when an ambient temperature is 85 degrees Fahrenheit and the thermal transfer apparatus was stored in a freezer with a temperature in a range of 5 degrees Fahrenheit to 25 degrees Fahrenheit for at least 12 hours.

Clause 27. The system of any of clauses 1-26, further comprising a bolster disposed on the first side.

Clause 28. A method comprising: disposing the thermal transfer apparatus of the thermal transfer system of any of clauses 1-27 in a freezer to cool the thermal transfer apparatus to a temperature of less than or equal to the second freezing point, thereby forming a cooled thermal transfer apparatus; and disposing the cooled thermal transfer apparatus in the cavity of the outer housing; and cooling an object placed on the first side.

Clause 29. A thermal transfer system comprising: an outer housing comprising a first side and a second side, the outer housing defining a cavity therein; a first layer positioned in the cavity, wherein the first layer has a first thickness of at least 0.5 inches and comprises a polyurethane foam; a second layer positioned in the cavity; and a thermal transfer apparatus removably positioned in the cavity and intermediate the first layer and the second layer, wherein the first layer is intermediate the thermal transfer apparatus and the first side, the thermal transfer apparatus comprising: a first container comprising a first wall, and a second wall connected to the first wall at ends of each wall, thereby defining a first enclosed cavity, at least four protrusions extending from the first wall into the first enclosed cavity towards the second wall and each protrusion comprising a third wall defining a second enclosed cavity, a first thermal transfer media positioned within the first enclosed cavity, and a second thermal transfer media positioned within each second enclosed cavity, wherein the first thermal transfer media has a first freezing point lower than a second freezing point of the second thermal transfer media.

Clause 30. A thermal transfer system comprising: an outer housing comprising a first side and a second side, the outer housing defining a cavity therein; a first layer positioned in the cavity, wherein the first layer has a first thickness of at least 0.5 inches and comprises a batting comprising polyester, cotton, or a combination of polyester and cotton; a second layer positioned in the cavity; and a thermal transfer apparatus removably positioned in the cavity and intermediate the first layer and the second layer, wherein the first layer is intermediate the thermal transfer apparatus and the first side, the thermal transfer apparatus comprising: a first container comprising a first wall, and a second wall connected to the first wall at ends of each wall, thereby defining a first enclosed cavity, at least four protrusions extending from the first wall into the first enclosed cavity towards the second wall and each protrusion comprising a third wall defining a second enclosed cavity, a first thermal transfer media positioned within the first enclosed cavity, and a second thermal transfer media positioned within each second enclosed cavity, wherein the first thermal transfer media has a first freezing point lower than a second freezing point of the second thermal transfer media.

Clause 31. A thermal transfer system comprising: an outer housing comprising a first side and a second side, the outer housing defining a cavity therein; a first layer positioned in the cavity, wherein the first layer has a first thickness of at least 0.5 inches and exhibits a thermal efficiency of no greater than 0.05 w/m*K; a second layer positioned in the cavity; and a thermal transfer apparatus removably positioned in the cavity and intermediate the first layer and the second layer, wherein the first layer is intermediate the thermal transfer apparatus and the first side.

In this specification, unless otherwise indicated, all numerical parameters are to be understood as being prefaced and modified in all instances by the term “about,” in which the numerical parameters possess the inherent variability characteristic of the underlying measurement techniques used to determine the numerical value of the parameter. At the very least, and not as an attempt to limit the application of the doctrine of equivalents to the scope of the claims, each numerical parameter described herein should at least be construed in light of the number of reported significant digits and by applying ordinary rounding techniques.

Also, any numerical range recited herein includes all sub-ranges subsumed within the recited range. For example, a range of “1 to 10” includes all sub-ranges between (and including) the recited minimum value of 1 and the recited maximum value of 10, that is, having a minimum value equal to or greater than 1 and a maximum value equal to or less than 10. Any maximum numerical limitation recited in this specification is intended to include all lower numerical limitations subsumed therein, and any minimum numerical limitation recited in this specification is intended to include all higher numerical limitations subsumed therein. Accordingly, Applicant reserves the right to amend this specification, including the claims, to expressly recite any sub-range subsumed within the ranges expressly recited. All such ranges are inherently described in this specification.

The grammatical articles “a”, “an”, and “the”, as used herein, are intended to include “at least one” or “one or more”, unless otherwise indicated, even if “at least one” or “one or more” is expressly used in certain instances. Thus, the foregoing grammatical articles are used herein to refer to one or more than one (i.e., to “at least one”) of the particular identified elements. Further, the use of a singular noun includes the plural, and the use of a plural noun includes the singular, unless the context of the usage requires otherwise.

As used herein, “intermediate” means that the referenced element is disposed between two elements but is not necessarily in contact with those elements. Accordingly, unless stated otherwise herein, an element that is “intermediate” a first element and a second element may or may not be adjacent to or in contact with the first and/or second elements, and other elements may be disposed between the intermediate element and the first and/or second elements.

One skilled in the art will recognize that the herein described thermal support, structures, operations/actions, and objects, and the discussion accompanying them, are used as examples for the sake of conceptual clarity and that various configuration modifications are contemplated. Consequently, as used herein, the specific examples/embodiments set forth and the accompanying discussion are intended to be representative of their more general classes. In general, use of any specific exemplar is intended to be representative of its class, and the non-inclusion of specific components, devices, apparatus, operations/actions, and objects should not be taken as limiting. While the present disclosure provides descriptions of various specific aspects for the purpose of illustrating various aspects of the present disclosure and/or its potential applications, it is understood that variations and modifications will occur to those skilled in the art. Accordingly, the invention or inventions described herein should be understood to be at least as broad as they are claimed and not as more narrowly defined by particular illustrative aspects provided herein.

What is claimed is:

1. A thermal transfer system comprising:
an outer housing comprising a first side and a second side, the outer housing defining a cavity therein;
a first layer positioned in the cavity, wherein the first layer has a first thickness of at least 0.5 inch and exhibits a thermal efficiency of no greater than 0.05 w/m*K;
a second layer positioned in the cavity; and
a thermal transfer apparatus removably positioned in the cavity and intermediate the first layer and the second layer, wherein the first layer is intermediate the thermal transfer apparatus and the first side, the thermal transfer apparatus comprising:
a first container comprising
a first wall, and
a second wall connected to the first wall at ends of each wall, thereby defining a first enclosed cavity,
at least four protrusions extending from the first wall into the first enclosed cavity towards the second wall and each protrusion comprising a third wall defining a second enclosed cavity,
a first thermal transfer media positioned within the first enclosed cavity, and
a second thermal transfer media positioned within each second enclosed cavity, wherein the first thermal transfer media has a first freezing point lower than a second freezing point of the second thermal transfer media.

2. The system of claim 1, wherein the first layer exhibits a thermal efficiency of no greater than 0.04 w/m*K.

3. The system of claim 1, wherein the first layer exhibits a thermal efficiency of no greater than 0.025 w/m*K.

4. The system of claim 1, wherein the second layer exhibits a thermal efficiency of no greater than 0.05 w/m*K.

5. The system of claim 1, wherein the second layer comprises a second thickness and the second thickness is greater than the first thickness.

6. The system of claim 1, wherein the second thickness is in a range of 2 inches to 4 inches and the first thickness is in a range of 1 inch to 3 inches.

7. The system of claim 1, wherein the first layer comprises an egg-crate shape or a substantially planar shape and the second layer comprises an egg-crate shape or a substantially planar shape.

8. The system of claim 1, wherein the first layer and the second layer, individually, comprise polyurethane, polyester, cotton, or a combination thereof.

9. The system of claim 1, wherein the first layer and the second layer, individually, comprise foam, batting, or a combination thereof.

10. The system of claim 1, wherein the second side comprises at least one slip resistant feature.

11. The system of claim 1, wherein the outer housing comprises fabric.

12. The system of claim 1, wherein the outer housing comprises a zipper, a hook and loop fastener, snaps, or a combination thereof.

13. The system of claim 1, wherein the thermal transfer apparatus is positioned within a bag.

14. The system of claim 1, wherein the at least four protrusions are arranged in a grid.

15. The system of claim 1, further comprising sixteen protrusions including the at least four protrusions, wherein the sixteen protrusions are arranged in four rows and four columns to form a first module.

16. The system of claim 15, further comprising at least two modules, including the first module and a second module.

17. The system of claim 1, wherein the first freezing point is lower than the second freezing point by at least 3 degrees Celsius and the second freezing point is in a range of −5 degrees Celsius to 5 degrees Celsius.

18. The system of claim 1, wherein the first thermal transfer media and the second thermal transfer media, individually, comprises water, glycol, gelatin, a hydrogel, an additive, or a combination thereof.

19. The system of claim 1, wherein the first wall, the second wall, and the third wall, each comprise plastic.

20. The system of claim 1, wherein the first wall, the second wall, and the third wall, each comprise a thickness in a range of 0.5 mm to 5 mm.

21. The system of claim 1, wherein the system is configured to maintain a temperature on the first side of no greater than 5 degrees Fahrenheit for at least 4 hours when an ambient temperature is 85 degrees Fahrenheit and the thermal transfer apparatus was stored in a freezer with a temperature in a range of 5 degrees Fahrenheit to 25 degrees Fahrenheit for at least 12 hours.

22. The system of claim 1, further comprising a bolster disposed on the first side.

23. A method comprising:
disposing the thermal transfer apparatus of the thermal transfer system of claim 1 in a freezer to cool the thermal transfer apparatus to a temperature of less than or equal to the second freezing point, thereby forming a cooled thermal transfer apparatus; and
disposing the cooled thermal transfer apparatus in the cavity of the outer housing; and
cooling an object placed on the first side.

24. A thermal transfer system comprising:
an outer housing comprising a first side and a second side, the outer housing defining a cavity therein;
a first layer positioned in the cavity, wherein the first layer has a first thickness of at least 0.5 inches and comprises a polyurethane foam, a batting comprising polyester, cotton, or a combination of polyester and cotton, or a combination of polyurethane foam and batting;
a second layer positioned in the cavity; and
a thermal transfer apparatus removably positioned in the cavity and intermediate the first layer and the second layer, wherein the first layer is intermediate the thermal transfer apparatus and the first side, the thermal transfer apparatus comprising:
a first container comprising
a first wall, and
a second wall connected to the first wall at ends of each wall, thereby defining a first enclosed cavity,
at least four protrusions extending from the first wall into the first enclosed cavity towards the second wall and each protrusion comprising a third wall defining a second enclosed cavity,
a first thermal transfer media positioned within the first enclosed cavity, and
a second thermal transfer media positioned within each second enclosed cavity, wherein the first thermal transfer media has a first freezing point lower than a second freezing point of the second thermal transfer media.

25. A thermal transfer system comprising:
an outer housing comprising a first side and a second side, the outer housing defining a cavity therein;
a first layer positioned in the cavity, wherein the first layer has a first thickness of at least 0.5 inches and exhibits a thermal efficiency of no greater than 0.05 w/m*K;
a second layer positioned in the cavity; and
a thermal transfer apparatus removably positioned in the cavity and intermediate the first layer and the second layer, wherein the first layer is intermediate the thermal transfer apparatus and the first side.
